(19) United States
(12) Patent Application Publication
(10) Pub. No.: US 2022/0047486 A1
(43) Pub. Date: Feb. 17, 2022

(54) COMPOSITION OF ACTIVE INGREDIENTS, FOR CARE AND SURFACE MODIFICATION OF HUMAN HAIR

(71) Applicant: Henkel AG & Co. KGaA, Duesseldorf (DE)

(72) Inventors: RENE KROHN, Norderstedt (DE); ERIK SCHULZE ZUR WIESCHE, Bielefeld (DE); TORSTEN LECHNER, Langenfeld (DE)

(73) Assignee: Henkel AG & Co. KGaA, Duesseldorf (DE)

(21) Appl. No.: 17/290,206

(22) PCT Filed: Oct. 31, 2019
(86) PCT No.: PCT/EP2019/079766

§ 371 Date: Apr. 29, 2021

(30) Foreign Application Priority Data
Oct. 31, 2018 (DE) 10 2018 127 181.6

(51) Int. Cl. A61K 8/58; A61K 8/44; A61K 8/65; A61K 8/64; A61K 8/898; A61K 8/49; A61Q 5/12

(52) U.S. Cl. CPC A61K 8/585; A61K 8/44; A61K 8/65; A61Q 5/12; A61K 8/898; A61K 8/4946; A61K 8/645

(57) ABSTRACT

The present disclosure relates to an active ingredient composition for the care and surface modification of human hair. In particular, the present disclosure relates to a cosmetic agent for treating a keratinous material comprising a) at least one organic silicon compound and b) at least one further component selected from the group consisting of an amino acid, an amino acid-derived polymer, a silanized amino acid and an amino acid-derived silanized polymer.

### CROSS-REFERENCE TO RELATED APPLICATION

This application is a U.S. National-Stage entry under 35 U.S.C. § 371 based on International Application No. PCT/EP2019/079766, filed Oct. 31, 2019, which was published under PCT Article 21(2) and which claims priority to German Application No. 10 2018127181.6, filed Oct. 31, 2018, which are all hereby incorporated in their entirety by reference.

### TECHNICAL FIELD

The present disclosure relates to cosmetic agents for treating a keratinous material, the agent comprising an organic silicon compound and another component selected from the group consisting of an amino acid, an amino acid-derived polymer, a silanized amino acid, and an amino acid-derived silanized polymer, and the use of the cosmetic agent.

### BACKGROUND

The exposure of hair to external chemicals from a variety of different sources poses challenges for the development of cosmetic care products. Air and water impurities have a detrimental effect on skin and hair. The most important air pollutants include polycyclic aromatic hydrocarbons, volatile organic compounds, nitrogen oxides (NOx), particles and cigarette smoke. The action of various atmospheric pollutants can be intensified in the presence of other atmospheric pollutants and under UV radiation action.

It is known that the toxicity of gaseous pollutants of air, such as sulfur dioxide, ozone and nitrogen oxides, is related in particular to their initiator activity for free radicals which cause damage to living organisms. Free radicals are metabolites that also occur naturally in the body. In large quantities, free radicals can promote irritation and inflammation and accelerate the process of aging. In this case, one speaks of “oxidative damage” Free radicals can also cause visible hair damage, for example, as a reduction in shine as well as grip and/or fading of hair color.

Particulate matter is a complex mixture containing metals, minerals, organic toxins, and/or biological materials. They can also promote the formation of free radicals.

Furthermore, often changing consumer demands for a certain hair texture are associated with recurring hair chemical stress. For example, hair colorings stress the hair, as a result of which special, intensive care may be necessary.

In the prior art, organosilicon compounds from the group of silanes comprising at least one hydroxy group and/or hydrolyzable group are described. Due to the presence of hydroxy groups and/or hydrolyzable groups, silanes are reactive substances that hydrolyze or oligomerize or polymerize in the presence of water. The oligomerization or polymerization of the silanes initiated by the presence of the water, when applied to a keratinous material, ultimately leads to the formation of a film that can provide a protective effect.

### BRIEF SUMMARY

In an exemplary embodiment, a cosmetic agent is provided for the treatment of a keratinous material. An exemplary cosmetic agent includes at least one organic silicon compound and at least one other component selected from the group consisting of an amino acid, an amino acid-derived polymer, a silanized amino acid, and an amino acid-derived silanized polymer.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The task underlying the present disclosure is to provide a product with an improved care and/or protection effect. In particular, the present disclosure was based on the task of providing a cosmetic agent which, after a hair treatment, enables a caring after-treatment which provides special care to stressed hair.

This problem is solved by a cosmetic agent for the treatment of a keratinous material, comprising:

a) at least one organic silicon compound; and
b) at least one other component selected from the group consisting of an amino acid, an amino acid-derived polymer, a silanized amino acid, and an amino acid-derived silanized polymer.

Keratinous material includes hair, skin, nails (such as fingernails and/or toenails). Wool, furs and feathers also fall under the definition of keratinous material.

Preferably, keratinous material is understood to be human hair, human skin and human nails, especially fingernails and toenails. In particular, keratinous material is understood to mean human hair, especially head and/or beard hair.

As a first ingredient, the cosmetic agent for treating a keratinous material contains at least one organic silicon compound. Preferred organic silicon compounds are selected from silanes having one, two or three silicon atoms, wherein the organic silicon compound comprises one or more hydroxyl groups and/or hydrolyzable groups per molecule.

Organic silicon compounds, alternatively called organosilicon compounds, are compounds which either have a direct silicon-carbon bond (Si—C) or in which the carbon is bonded to the silicon atom via an oxygen, nitrogen or sulfur atom. The organic silicon compounds are compounds containing one to three silicon atoms. Organic silicon compounds preferably contain one or two silicon atoms.

According to IUPAC rules, the term silane stands for a group of chemical compounds based on a silicon skeleton and hydrogen. In organic silanes, the hydrogen atoms are completely or partially replaced by organic groups such as (substituted) alkyl groups and/or alkoxy groups. In organic silanes, some of the hydrogen atoms may also be replaced by hydroxy groups.

The agent for treating a keratinous material contains at least one organic silicon compound preferably selected from silanes having one, two or three silicon atoms, wherein the organic silicon compound comprises one or more hydroxyl groups or hydrolyzable groups per molecule.

In a particularly preferred embodiment, the agent for treating a keratinous material comprises at least one organic silicon compound selected from silanes having one, two or three silicon atoms, the organic silicon compound further comprising one or more basic groups and one or more hydroxyl groups or hydrolyzable groups per molecule.

This basic group can be, for example, an amino group, an alkylamino group or a dialkylamino group, which is preferably connected to a silicon atom via a linker. The basic group is preferably an amino group, a C₁-C₆ alkylamino group or a di(C₁-C₆) alkylamino group.

The hydrolyzable group(s) is (are) preferably a C₁-C₆ alkoxy group, especially an ethoxy group or a methoxy group. It is preferred when the hydrolyzable group is directly bonded to the silicon atom. For example, if the hydrolyzable group is an ethoxy group, the organic silicon compound preferably contains a structural unit R′R″R′″Si—O—CH₂—CH₃. The residues R′, R″ and R′″ represent the three remaining free valences of the silicon atom.

Particularly good results were obtained when the agent for treating a keratinous material contains at least one organic silicon compound of formula (I) and/or (II).

The compounds of formulae (I) and (II) are organic silicon compounds selected from silanes having one, two or three silicon atoms, the organic silicon compound comprising one or more hydroxyl groups and/or hydrolysable groups per molecule.

In another very particularly preferred embodiment, the agent for treating a keratinous material comprises at least one organic silicon compound of formula (I) and/or (II),

R₁R₂N-L-Si(OR₃)ₐ(R₄)_{b} (I),
where

R₁, R₂ both represent a hydrogen atom,

L represents a linear, divalent C₁-C₆-alkylene group, preferably a propylene group (—CH₂—CH₂—CH₂—) or an ethylene group (—CH₂—CH₂—),

R₃, R₄ independently represent a methyl group or an ethyl group,

a stands for the number 3, and

b stands for the number 0.

(R₅O)_{c}(R₆)_{d}Si-(A)ₑ-[NR₇-(A′)]_{f}—[O-(A″)]_{g}—[NR₈-(A′″)]ₕ—Si(R₆′)_{d′}(OR₅′)_{c′} (II),
where

R5, R5′, R5″ independently represent a hydrogen atom or a C₁-C₆ alkyl group,

R6, R6′ and R6″ independently represent a C₁-C₆ alkyl group,

A, A′, A″, A′″ and A″″ independently represent a linear or branched divalent C₁-C₂₀ alkylene group, and

R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III)

-(A″″)-Si(R₆″)_{d}″(OR₅″)_{c}″ (III),

where
c, stands for an integer from 1 to 3,
d stands for the integer 3-c,
c′ stands for an integer from 1 to 3,
d′ stands for the integer 3-c′,
c″ stands for an integer from 1 to 3,
d″ stands for the integer 3-c″,
e stands for 0 or 1,
f stands for 0 or 1,
g stands for 0 or 1, and
h stands for 0 or 1,
provided that at least one of e, f, g and h is different from 0.

The substituents R₁, R₂, R₃, R₄, R₅, R₅′, R₅″, R₆, R₆′, R₆″, R₇, R₈, L, A′, A″″ and A′″″ in the compounds of formula (I) and (II) are explained below as examples:

Examples of a C₁-C₆ alkyl group are the groups methyl, ethyl, propyl, isopropyl, n-butyl, s-butyl and t-butyl, n-pentyl and n-hexyl. Propyl, ethyl and methyl are preferred alkyl radicals. Examples of a C₂-C₆ alkenyl group are vinyl, allyl, but-2-enyl, but-3-enyl and isobutenyl, preferred C₂-C₆ alkenyl radicals are vinyl and allyl. Preferred examples of a hydroxy C₁-C₆ alkyl group are a hydroxymethyl, a 2-hydroxyethyl, a 2-hydroxypropyl, a 3-hydroxypropyl, a 4-hydroxybutyl group, a 5-hydroxypentyl and a 6-hydroxyhexyl group; a 2-hydroxyethyl group is particularly preferred. Examples of an amino C₁-C₆ alkyl group are the aminomethyl group, the 2-aminoethyl group, the 3-aminopropyl group. The 2-aminoethyl group is particularly preferred. Examples of a linear divalent C₁-C₂₀ alkylene group include the methylene group (—CH₂),), the ethylene group (—CH₂—CH₂—), the propylene group (—CH₂—CH₂—CH₂—) and the butylene group (—CH₂—CH₂—CH₂—). The propylene group (—CH₂—CH₂—CH₂—) is particularly preferred. From a chain length of 3 C atoms, divalent alkylene groups can also be branched. Examples of branched divalent C₃-C₂₀ alkylene groups are (—CH₂—CH(CH₃)—) and (—CH₂—CH(CH₃)—CH₂—).

In the organic silicon compounds of the formula (I)

R₁R₂N-L-Si(OR₃)ₐ(R₄)_{b} (I),

the radicals R₁ and R₂ independently of one another represent a hydrogen atom or a C₁-C₆ alkyl group. In particular, the radicals R₁ and R₂ both represent a hydrogen atom.

In the middle part of the organic silicon compound is the structural unit or the linker -L-, which stands for a linear or branched, divalent C₁-C₂₀ alkylene group.

Preferably, -L- represents a linear, divalent C₁-C₂₀ alkylene group. More preferably -L- stands for a linear divalent C₁-C₆ alkylene group. Particularly preferred -L stands for a methylene group (CH₂—), an ethylene group (—CH₂—CH₂—), propylene group (—CH₂—CH₂—CH₂—) or butylene (—CH₂—CH₂—CH₂—CH₂—). In particular, L stands for a propylene group (—CH₂—CH₂—CH₂—)

The organic silicon compounds of formula (I)

R₁R₂N-L-Si(OR₃)ₐ(R₄)_{b} (I),

one end of each carries the silicon-containing group —Si(OR₃)ₐ(R₄)_{b}

In the terminal structural unit —Si(OR₃)ₐ(R₄)_{b}, R₃ is hydrogen or C₁-C₆ alkyl group, and R₄ is C₁-C₆ alkyl group. In particular, R3 and R3 independently of each other represent a methyl group or an ethyl group.

Here a stands for an integer from 1 to 3, and b stands for the integer 3-a. If a stands for the number 3, then b is equal to 0. If a stands for the number 2, then b is equal to 1. If a stands for the number 1, then b is equal to 2.

The best protection against the negative effects of water and/or air pollution (“anti-pollution” effect) and the best care of stressed hair could be obtained if the agent for treating a keratinous material contains at least one organic silicon compound of formula (I) in which the radicals R₃, R₄ independently represent a methyl group or an ethyl group.

Particularly well-suited organic silicon compounds of formula (I) are

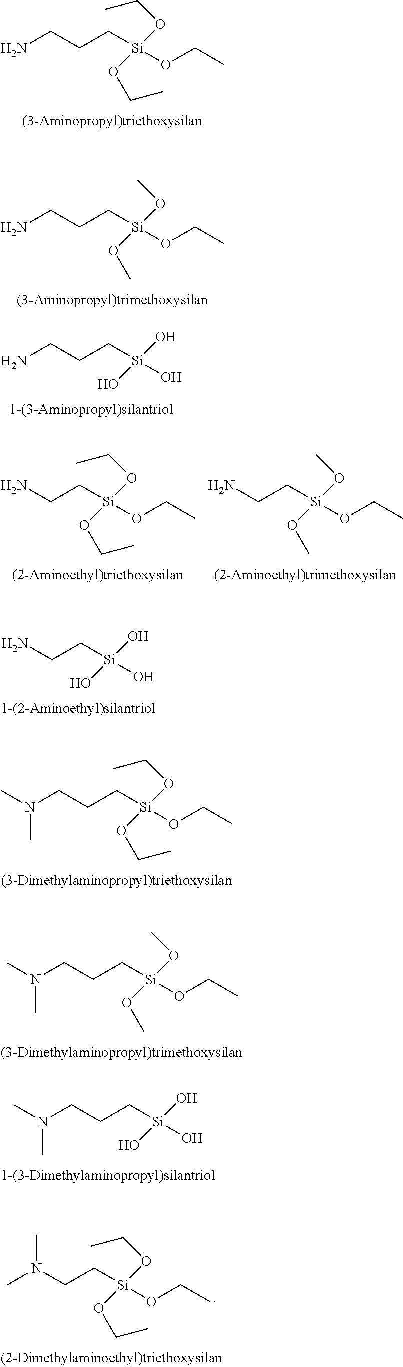

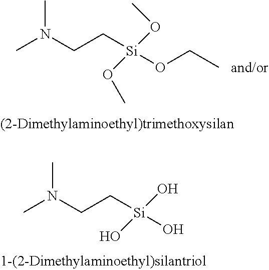

The aforementioned organic silicon compound of formula (I) is commercially available. (3-aminopropyl)trimethoxysilane, for example, can be purchased from Sigma-Aldrich. (3-Aminopropyl)triethoxysilane is also commercially available from Sigma-Aldrich.

In another embodiment, the composition for treating a keratinous material comprises at least one organic silicon compound of formula (II)

(R₅O)_{c}(R₆)_{d}Si-(A)ₑ-[NR₇-(A′)]_{f}—[O-(A″)]_{g}—[NR₈-(A′″)]ₕ—Si(R₆′)_{d′}(OR₅′)_{c′} (II),

The organosilicon compounds of formula (II) as contemplated herein each carry the silicon-containing groups (R₅O)_{c}(R₆)_{d}Si— and —Si(R₆′)_{d′}(OR₅′)_{c} at both ends.

In the central part of the molecule of formula (II) there are the groups -(A)ₑ- and —[NR₇-(A′)]_{f}- and —[O-(A″)]_{g}- and —[NR₈-(A′″)]ₕ-. Here, each of the radicals e, f, g and h can independently of one another stand for the number 0 or 1, with the proviso that at least one of the radicals e, f, g and h is different from 0. In other words, an organic silicon compound of formula (II) as contemplated herein contains at least one grouping from the group consisting of -(A)- and —[NR₇-(A′)]- and —[O-(A″)]- and —[NR₈-(A′″)]-.

In the two terminal structural units (R₅O)_{c}(R₆)_{d}Sii- and —Si(R₆′)_{d′}(OR₅′)ₑ, the radicals R5, R5′, R5″ independently of one another represent a hydrogen atom or a C₁-C₆ alkyl group. The radicals R6, R6′ and R6″ independently represent a C₁-C₆ alkyl group.

Here a stands for an integer from 1 to 3, and d stands for the integer 3-c. If c stands for the number 3, then d is equal to 0. If c stands for the number 2, then d is equal to 1. If c stands for the number 1, then d is equal to 2.

Analogously c′ stands for a whole number from 1 to 3, and d′ stands for the whole number 3-c′. If c′ stands for the number 3, then d′ is 0. If c′ stands for the number 2, then d′ is 1. If c′ stands for the number 1, then d′ is 2.

A very high anti-pollution effect of the agent for the treatment of a keratin material could be obtained if the residues c and c′ both represent the number 3. In this case d and d′ both stand for the number 0.

In a further preferred embodiment, the agent for treating a keratin material contains at least one organic silicon compound of formula (II)

(R₅O)_{c}(R₆)_{d}Si-(A)ₑ-[NR₇-(A′)]_{f}—[O-(A″)]_{g}—[NR₈-(A′″)]ₕ—Si(R₆′)_{d′}(OR₅′)_{c′} (II),

where

R5 and R5′ independently represent a methyl group or an ethyl group,

c and c′ both stand for the number 3, and

d and d′ both stand for the number 0.

If c and c′ are both the number 3 and d and d′ are both the number 0, the organic silicon compound of the present disclosure corresponds to formula (IIa)

(R₅O)₃Si-(A)ₑ-[NR₇-(A′)]_{f}—[O-(A″)]_{g}—[NR₈-(A′″)]ₕ—Si(OR₅′)₃ (IIa).

The radicals e, f, g and h can independently stand for the number 0 or 1, whereby at least one radical from e, f, g and h is different from zero. The abbreviations e, f, g and h thus define which of the groupings -(A)e- and -[NR7-(A′)]f- and —[O-(A″)]g- and -[NR8-(A′″)]h- are located in the middle part of the organic silicon compound of formula (II).

In this context, the presence of certain groups has proved to be particularly advantageous in terms of increasing the “anti-pollution” effect. Particularly good results were obtained when at least two of the residues e, f, g and h stand for the number 1. Especially preferred e and f both stand for the number 1. Furthermore, g and h both stand for the number 0.

If e and f both stand for the number 1 and g and h both stand for the number 0, the organic silicon compound as contemplated herein corresponds to formula (IIb)

(R₅O)_{c}(R₆)_{d}Si-(A)-[NR₇-(A′)]—Si(R₆′)_{d′}(OR₅′)_{e′} (IIb).

The radicals A, A′, A″, A′″ and A″″ independently represent a linear or branched divalent C₁-C₂₀ alkylene group. Preferably the radicals A, A′, A″, A′″ and A″″ independently of one another represent a linear, divalent C₁-C₂₀ alkylene group. Further preferably the radicals A, A′, A″, A′″ and A″″ independently represent a linear divalent C₁-C₆ alkylene group. In particular, the radicals A, A′, A″, A′″ and A″″ independently of one another represent a methylene group (—CH₂—), an ethylene group (—CH₂—CH₂—), a propylene group (—CH₂—CH₂—CH₂—) or a butylene group (—CH₂—CH₂—CH₂—CH₂—). In particular, the residues A, A′, A″, A′″ and A″″ stand for a propylene group (—CH₂—CH₂—CH₂—).

When the radical f represents the number 1, the organic silicon compound of formula (II) contains a structural grouping —[NR7-(A′)]-.

When the radical h represents the number 1, the organic silicon compound of formula (II) contains a structural grouping —[NR8-(A″)]-.

Wherein R₇ and R₈ independently represent a hydrogen atom, a C₁-C₆ alkyl group, a hydroxy-C₁-C₆ alkyl group, a C₂-C₆ alkenyl group, an amino-C₁-C₆ alkyl group or a group of the formula (III)

-(A″″)-Si(R₆″)_{d}″(OR₅″)_{c″} (III),

Very preferably, R₇ and R₈ independently represent a hydrogen atom, a methyl group, a 2-hydroxyethyl group, a 2-alkenyl group, a 2-aminoethyl group or a group of formula (III).

If the radical f represents the number 1 and the radical h represents the number 0, the organic silicon compound as contemplated herein contains the grouping [NR₇-(A′)] but not the grouping —[NR₈-(A′″)]. If the residue R7 is a grouping of formula (III), the agent for treating a keratin material contains an organic silicon compound with 3 reactive silane groups.

In a further preferred embodiment, the agent for treating a keratin material contains at least one organic silicon compound of formula (II)

(R₅O)_{c}(R₆)_{d}Si-(A)ₑ-[NR₇-(A′)]_{f}—[O-(A″)]_{g}—[NR₈-(A′″)]ₕ—Si(R₆′)_{d′}(OR₅′)_{c′} (II),

where

e and f both stand for the number 1,

g and h both stand for the number 0,

A and A′ independently of one another represent a linear, divalent C₁-C₆ alkylene group, and

R7 represents a hydrogen atom, a methyl group, a 2-hydroxyethyl group, a 2-alkenyl group, a 2-aminoethyl group, or a group of formula (III).

In another preferred embodiment, the agent for treating a keratin material contains at least one organic silicon compound of formula (II), wherein

e and f both stand for the number 1,

g and h both stand for the number 0,

A and A′ independently of one another represent a methylene group (—CH₂—), an ethylene group (—CH₂—CH₂—) or a propylene group (—CH₂—CH₂—CH₂),

and

R₇ represents a hydrogen atom, a methyl group, a 2-hydroxyethyl group, a 2-alkenyl group, a 2-aminoethyl group, or a group of formula (III).

Organic silicon compounds of formula (II) that are suitable for solving the problem are

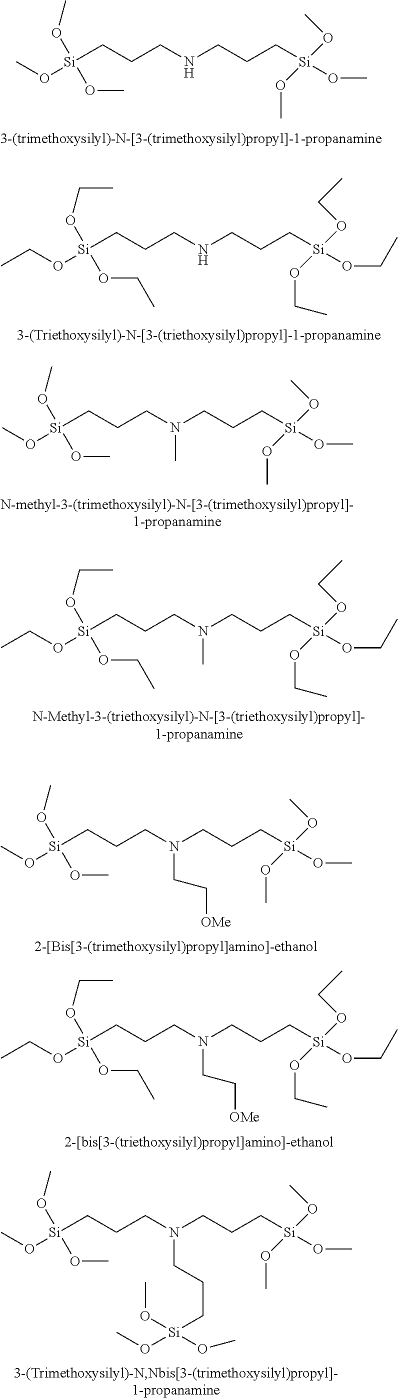

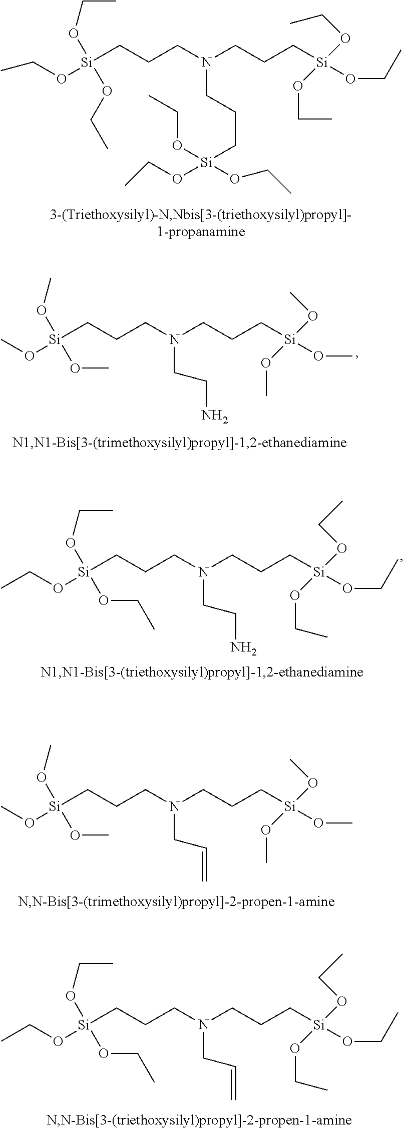

The aforementioned organic silicon compounds of formula (II) are commercially available.

Bis(trimethoxysilylpropyl)amines with the CAS number 82985-35-1 can be purchased from Sigma-Aldrich.

Bis[3-(triethoxysilyl)propyl]amine, also known as 3-(triethoxysilyl)-N-[3 (triethoxysilyl)propyl]-1-propanamine, having the CAS number 13497-18-2, can be purchased, for example, from Sigma-Aldrich or is commercially available under the product name Dynasylan 1122 from Evonik.

N-methyl-3-(trimethoxysilyl)-N-[3-(trimethoxysilyl)propyl]-1-propanamine is alternatively referred to as bis(3-trimethoxysilylpropyl)-N-methylamine and can be purchased commercially from Sigma-Aldrich or Fluorochem.

3-(triethoxysilyl)-N,N-bis[3-(triethoxysilyl)propyl]-1-propanamine with the CAS number 18784-74-2 can be purchased for example from Fluorochem or Sigma-Aldrich.

It has also been found to be advantageous if the agent for treating a keratinous material applied to the hair contains at least one organic silicon compound of formula (IV)

R₉Si(OR₁₀)ₖ(R₁₁)ₘ (IV).

The compounds of formula (IV) are organic silicon compounds selected from silanes having one, two or three silicon atoms, the organic silicon compound comprising one or more hydroxyl groups and/or hydrolysable groups per molecule.

The organic silicon compound(s) of formula (IV) may also be referred to as silanes of the alkylalkoxysilane or alkylhydroxysilane type,

R₉Si(OR₁₀)ₖ(R₁₁)ₘ (IV).

where

R₉ represents a C₁-C₁₂ alkyl group,

R₁₀ represents a hydrogen atom or a C₁-C₆ alkyl group,

R₁₁ represents a C₁-C₆ alkyl group

k is an integer from 1 to 3, and

m stands for the integer 3-k.

In a further preferred embodiment, the agent for treating a keratin material contains, in addition to the organic silicon compounds of formula (I), at least one further organic silicon compound of formula (IV)

R₉Si(OR₁₀)ₖ(R₁₁)ₘ (IV).

where

R₉ represents a C₁-C₁₂ alkyl group,

R₁₀ represents a hydrogen atom or a C₁-C₆ alkyl group,

R₁₁ represents a C₁-C₆ alkyl group

k is an integer from 1 to 3, and

m stands for the integer 3-k.

In a likewise preferred embodiment, the agent for treating a keratin material contains at least one further organic silicon compound of formula (IV) in addition to the organic silicon compounds of formula (II)

R₉Si(OR₁₀)ₖ(R₁₁)ₘ (IV).

where

R₉ represents a C₁-C₁₂, alkyl group,

R₁₀ represents a hydrogen atom or a C₁-C₆ alkyl group,

R₁₁ represents a C₁-C₆ alkyl group

k is an integer from 1 to 3, and

m stands for the integer 3-k.

In a further preferred embodiment, the agent for treating a keratin material contains, in addition to the organic silicon compounds of formula (I) and (II), at least one further organic silicon compound of formula (IV)

R₉Si(OR₁₀)ₖ(R₁₁)ₘ (IV).

where

R₉ represents a C₁-C₁₂, alkyl group,

R₁₀ represents a hydrogen atom or a C₁-C₆ alkyl group,

R₁₁ represents a C₁-C₆ alkyl group

k is an integer from 1 to 3, and

m stands for the integer 3-k.

In the organic silicon compounds of formula (IV), the radical R₉ represents a C₁-C₁₂, alkyl group. This C₁-C₁₂ alkyl group is saturated and can be linear or branched. Preferably, R₉ stands for a linear C₁-C₈ alkyl group. Preferably, R₉ stands for a methyl group, an ethyl group, an n-propyl group, an n-butyl group, an n-pentyl group, an n-hexyl group, an n-octyl group or an n-dodecyl group. Particularly preferably, R₉ represents a methyl group, an ethyl group or an n-octyl group.

In the organic silicon compounds of formula (IV), the radical R₁₀ represents a hydrogen atom or a C₁-C₆ alkyl group. It is particularly preferred that R₁₀ represents a methyl group or an ethyl group.

In the organic silicon compounds of formula (IV), the radical R₁₁ represents a C₁-C₆ alkyl group. It is particularly preferred that R₁₁ represents a methyl group or an ethyl group.

Furthermore, k stands for a whole number from about 1 to about 3, and m stands for the whole number 3-k. If k stands for the number 3, then m is equal to 0. If k stands for the number 2, then m is equal to 1. If k stands for the number 1, then m is equal to 2.

A very high “anti-pollution” effect could be obtained if the agent for treating a keratinous material contains at least one organic silicon compound of formula (IV) in which the radical k represents the number 3. In this case, the rest m stands for the number 0.

Organic silicon compounds of the formula (IV) that are particularly suitable for solving the problem are

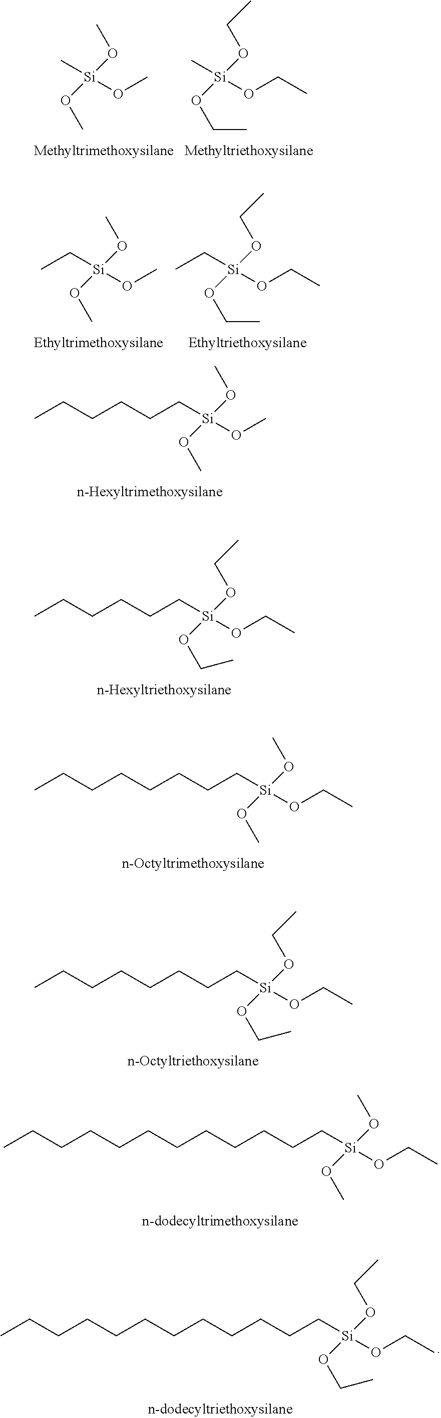

and also propyltrimethoxysilane, propyltriethoxysilane, octadecyltrimethoxysilane and/or octadecyltriethoxysilane.

The organic silicon compounds described above are reactive compounds.

In this context, it has been found to be particularly preferred if the agent contains as organic silicon compound (3-aminopropyl)triethoxysilane, i.e., an aminopropyltriethoxysilane (AMEO), and/or 3-(triethoxysilyl)-N-[3-(triethoxysilyl)propyl]-1-propanamine, i.e. a bis(triethoxysilylpropyl)amine

It was found that particularly stable and uniform films could be obtained on the keratinous material even when the agent contained two structurally different organic silicon compounds.

In a preferred embodiment, an agent comprises at least one organic silicone compound of formula (I) and at least one organic silicone compound of formula (IV).

In an explicitly very particularly preferred embodiment, an agent contains at least one organic silicone compound of formula (I) selected from the group consisting of (3-aminopropyl)triethoxysilane and (3-aminopropyl)trimethoxysilane, and additionally containing at least one organic silicone compound of formula (IV) selected from the group consisting of methyltrimethoxysilane, methyltriethoxysilane, ethyltrimethoxysilane, ethyltriethoxysilane, propyltrimethoxysilane, propyltriethoxysilane, hexyltrimethoxysilane, and hexyltriethoxysilane.

In another preferred embodiment, an agent is characterized in that the agent-contains, based on the total weight of the agent-:
0.5 to 5 weight % of at least one first organic silicon compound selected from the group of (3-aminopropyl)trimethoxysilane, (3-aminopropyl)triethoxysilane, (2-aminoethyl)trimethoxysilane, (2-aminoethyl)triethoxysilane, (3-dimethylaminopropyl)trimethoxysilane, (3-dimethylaminopropyl)triethoxysilane (2-dimethylaminoethyl)trimethoxysilane, and (2-dimethylaminoethyl)triethoxysilane, and
3.2 to 10 wt. % of at least one second organic silicon compound selected from the group consisting of methyltrimethoxysilane, methyltriethoxysilane, ethyltrimethoxysilane, ethyltriethoxysilane, propyltrimethoxysilane, propyltriethoxysilane, hexyltrimethoxysilane, hexyltriethoxysilane, octyltrimethoxysilane, octyltriethoxysilane, dodecyltrimethoxysilane, dodecyltriethoxysilane, octadecyltrimethoxysilane, and octadecyltriethoxysilane.

According to preferred embodiments, the organic silicon compound of formula (I), in particular the (3-aminopropyl)triethoxysilane, is present in an amount of from about 0.01 to about 10 wt. %, preferably from about 0.02 to about 8% by weight, more preferably from about 0.05 to about 6% by weight, most preferably from about 0.1 to about 4% by weight, based on the total weight of the cosmetic agent, in the cosmetic agent, and/or the organic silicon compound of the formula (II), in particular the 3-(triethoxysilyl)-N-[3-(triethoxysilyl)propyl]-1-propanamine, is present in an amount of from about 0.01 to about 10% by weight, preferably from about 0.02% to about %, preferably from about 0.02 to about 9% by weight, more preferably from about 0.05 to about 8% by weight, most preferably from about 0.1 to about 7% by weight, based on the total weight of the cosmetic agent, in the cosmetic agent.

In the context of embodiments herein, figures in wt.-% are—unless otherwise stated—always based on the total weight of the cosmetic agent.

Even the addition of small amounts of water leads to hydrolysis in the case of organic silicon compounds having at least one hydrolyzable group. The hydrolysis products and/or organic silicon compounds having at least one hydroxyl group can react with one another in a condensation reaction. For this reason, both the organosilicon compounds having at least one hydrolyzable group and their hydrolysis and/or condensation products may be present in the composition. When organosilicon compounds having at least one hydroxyl group are used, both the organic silicon compounds having at least one hydroxyl group and their condensation products may be present in the composition.

A condensation product is understood to be a product formed by the reaction of at least two organic silicon compounds each having at least one hydroxyl group or hydrolyzable group per molecule with elimination of water and/or with elimination of an alkanol. The condensation products can be, for example, dimers, but also trimers or oligomers, with the condensation products being in equilibrium with the monomers. Depending on the amount of water used or consumed in the hydrolysis, the equilibrium of monomeric organic silicon compounds shifts to condensation product.

As a second ingredient, the cosmetic agent for treating a keratinous material contains a further component b) selected from the group consisting of an amino acid, a polymer derived from an amino acid, a silanized amino acid and a silanized polymer derived from an amino acid. In the course of the work leading to the present disclosure, it has been found that, in order to achieve a particularly good care effect, it is particularly advantageous if the organic silicon compounds, for example (3-aminopropyl)triethoxysilane, i.e. an aminopropyltriethoxysilane (AMEO), or for example 3-(triethoxysilyl)-N-[3-(triethoxysilyl)propyl]-1-propanamine, i.e., a bis(triethoxysilylpropyl)amine, is combined with an amino acid, a polymer derived from an amino acid, a silanized amino acid or a silanized polymer derived from an amino acid.

It has been found that the combination of an amino acid, an amino acid-derived polymer, a silanized amino acid or an amino acid-derived silanized polymer, on the one hand, and the aminopropyltriethoxysilane (AMEO) and/or the bis(triethoxysilylpropyl)amine, on the other hand, is particularly powerful in terms of maintenance. The hair surface is hydrophobized, reducing frizz. The hair becomes softer and even with a high chemical stress, the hair can still be shaped better.

According to a preferred embodiment, the one or more components b) selected from the group consisting of an amino acid, an amino acid-derived polymer, a silanized amino acid, and an amino acid-derived silanized polymer are present in a total amount in the cosmetic composition that is from about 0.01 to about 10% by weight, preferably from about 0.25 to about 8% by weight, more preferably from about 0.5 to about 6% by weight.

An amino acid is initially understood to be a carboxylic acid that carries a primary amino group on the alpha-carbonyl carbon. Carboxylic acids with a total number of C atoms of C2-20, more preferably of C2-15, particularly preferably of C2-10 are preferred. According to a preferred embodiment, the amino acid is selected from the group consisting of arginine, histidine, proline, cysteine, and lanthionine.

Alternatively, the further component b) may represent a silanized amino acid. A silanized amino acid is a reaction product of an amino acid and a silane. Preferably, the amino acids subjected to silanization and used as silanized amino acids are selected from the group consisting of arginine, histidine, proline, cystine, and lanthionine.

In particular, when (silanized) amino acids are used as further component b), it is preferred if the pH of the cosmetic agent is in the range from about 1.5 to about 8, preferably from about 2 to about 7, more preferably from about 2.5 to about 6, most preferably from about 3 to about 5.

According to a preferred embodiment, the amino acid derived polymer is a protein hydrolysate. Even more preferably, component b) is elastin, collagen, keratin, silk, milk protein, soybean, almond, pea, rice, tomato, potato, wheat protein hydrolysates, or salts thereof, in particular keratin protein hydrolysates, wheat protein hydrolysates or salts thereof. Protein hydrolysates are product mixtures obtained by acidic, basic or enzymatic catalysed degradation of proteins. Protein hydrolysates of both plant and animal origin can be used.

According to a preferred embodiment, the silanized polymer derived from an amino acid is a silanized protein hydrolysate of the formula (PH-I)

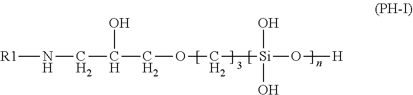

or the formula (PH-II)

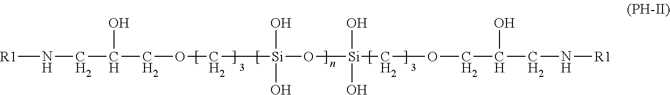

where n is an integer from 1 to 100, preferably from 1 to 50, more preferably from 1 to 20, particularly preferably from 1 to 10, even more preferably from 1 to 5, and most preferably from 1 to 3, and R1 is a polypeptide chain of the protein hydrolysate used. In this preferred embodiment, the protein hydrolysates forming the polypeptide chain may be derived from different amino acids. Particularly preferred polypeptide chains were based on cysteine.

The silanized protein hydrolysate or the silanized amino acid preferably has at least one hydroxy function at the silicon atom but may have a maximum of three hydroxy functions.

In very particularly preferred embodiments, the further component b) is selected from the group consisting of Cystine Bis-PG-Propyl Silanetriol (INCI) and Hydrolyzed Wheat Protein PG-Propyl Silanetriol (INCI). Such products are available for sale, for example, from Croda under the trade names Crodasone® W, Crodasone® W PF, Keravis® PE or Crodasone® Cystine.

In particular, the features for treating a keratinous material may comprise a feature for cleaning a keratinous material, a feature for maintaining a keratinous material, a feature for maintaining and cleaning a keratinous material, and/or a feature for temporarily reshaping a keratinous material.

Further ingredients of the hair treatment products are described below, which may be contained in the products in addition to the mandatory ingredients described above.

It may be preferred that the agent for treating a keratinous material further comprises from about 0.001 to about 20% by weight of at least one quaternary compound. This applies in particular to agents for the care of a keratinous material and agents for the care and cleaning of a keratinous material.

It is preferred that the at least one quaternary compound is selected from at least one of the groups consisting of:

i) the monoalkylquats, and/or
ii) the esterquats, and/or
iii) the quaternary imidazolines of formula (Tkat2),

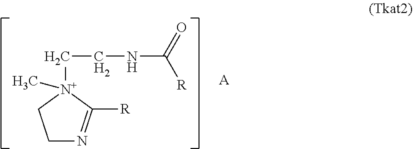

in which the radicals R independently of one another each represent a saturated or unsaturated, linear or branched hydrocarbon radical having a chain length of 8 to 30 carbon atoms and A represents a physiologically tolerated anion, and/or

iv) of the amidoamines and/or cationized amidoamines, and/or
v) Poly(methacryloyloxyethyltrimethylammonium compounds), and/or;
vi) quaternized cellulose derivatives, in particular polyquaternium 10, polyquaternium-24, polyquaternium-27, polyquaternium-67, polyquaternium-72, and/or
vii) cationic alkyl polyglycosides, and/or
viii) cationized honey, and/or
ix) cationic guar derivatives, and/or
x) chitosan, and/or
xi) polymeric dimethyldiallylammonium salts and copolymers thereof with esters and amides of acrylic acid and methacrylic acid, in particular polyquaternium-7, and/or
xii) copolymers of vinylpyrrolidone with quaternized derivatives of dialkylaminoalkyl acrylate and methacrylate, especially polyquaternium-11, and/or
xiii) vinylpyrrolidone-vinylimidazolium methochloride copolymers, in particular polyquaternium-16, and/or
xiv) quaternized polyvinyl alcohol, and/or
xv) polyquaternium-74,
and mixtures thereof.

It is particularly preferred that the hair treatment composition contains a cationic homopolymer falling under the INCI designation polyquaternium-37 as quaternary compounds.

It may be preferred that the agent for treating a keratinous material further comprises a strengthening compound, preferably selected from the group consisting of waxes, synthetic polymers and mixtures thereof.

In order to meet the different requirements for agents for the treatment of a keratinous material in the form of an agent for the temporary reshaping of a keratinous material (=styling agent), a large number of synthetic polymers have already been developed as strengthening compounds which can be used in an agent for the treatment of a keratinous material. Alternatively or additionally, waxes are used as strengthening compounds. Ideally, the polymers and/or waxes, when applied to the keratinous material, result in a polymer film or sheet that, on the one hand, gives the hairstyle a strong hold, but, on the other hand, is sufficiently flexible not to break when stressed.

The synthetic polymers can be divided into cationic, anionic, nonionic and amphoteric strengthening polymers.

Suitable synthetic polymers include, for example, polymers with the following INCI names: Acrylamide/Ammonium Acrylate Copolymer, Acrylamides/DMAPA Acrylates/Methoxy PEG Methacrylate Copolymer, Acrylamidopropyltrimonium Chloride/Acrylamide Copolymer, Acrylamidopropyltrimonium Chloride/Acrylates Copolymer, Acrylates/Acetoacetoxyethyl Methacrylate Copolymer, Acrylates/Acrylamide Copolymer, Acrylates/Ammonium Methacrylate Copolymer, Acrylates/t-Butylacrylamide Copolymer, Acrylates Copolymer, Acrylates/C1-2 Succinates/Hydroxyacrylates Copolymer, Acrylates/Lauryl Acrylate/Stearyl Acrylate/Ethylamine Oxide Methacrylate Copolymer, Acrylates/Octylacrylamide Copolymer, Acrylates/Octylacrylamide/Diphenyl Amodimethicone Copolymer, Acrylates/Stearyl Acrylate/Ethylamine Oxide Methacrylate Copolymer, Acrylates/VA Copolymer, Acrylates/Hydroxyesters Acrylates Copolymer, Acrylates/VP Copolymer, Adipic Acid/Diethylenetriamine Copolymer, Adipic Acid/Dimethylaminohydroxypropyl Diethylenetriamine Copolymer, Adipic Acid/Epoxypropyl Diethylenetriamine Copolymer, Adipic Acid/Isophthalic Acid/Neopentyl Glycol/Trimethylolpropane Copolymer, Allyl Stearate/VA Copolymer, Aminoethylacrylate Phosphate/Acrylates Copolymer, Aminoethylpropanediol-Acrylates/Acrylamide Copolymer, Aminoethylpropanediol-AMPD-Acrylates/Diacetoneacrylamide Copolymer, Ammonium VA/Acrylates Copolymer, AMPD-Acrylates/Diacetoneacrylamide Copolymer, AMP-Acrylates/Allyl Methacrylate Copolymer, AMP-Acrylates/C1-18 Alkyl Acrylates/C1-8 Alkyl Acrylamide Copolymer, AMP-Acrylates/Diacetoneacrylamide Copolymer, AMP-Acrylates/Dimethylaminoethylmethacrylate Copolymer, Bacillus/Rice Bran Extract/Soybean Extract Ferment Filtrate, Bis-Butyloxyamodimethicone/PEG-60 Copolymer, Butyl Acrylate/Ethylhexyl Methacrylate Copolymer, Butyl Acrylate/Hydroxypropyl Dimethicone Acrylate Copolymer, Butylated PVP, Butyl Ester of Ethylene/MA Copolymer, Butyl Ester of PVM/MA Copolymer, Calcium/Sodium PVM/MA Copolymer, Corn Starch/Acrylamide/Sodium Acrylate Copolymer, Diethylene Glycolamine/Epichlorohydrin/Piperazine Copolymer, Dimethicone Crosspolymer, Diphenyl Amodimethicone, Ethyl Ester of PVM/MA Copolymer, Hydrolyzed Wheat Protein/PVP Crosspolymer, Isobutylene/Ethylmaleimide/Hydroxyethylmaleimide Copolymer, Isobutylene/MA Copolymer, Isobutylmethacrylate/Bis-Hydroxypropyl Dimethicone Acrylate Copolymer, Isopropyl Ester of PVM/MA Copolymer, Lauryl Acrylate Crosspolymer, Lauryl Methacrylate/Glycol Dimethacrylate Crosspolymer, MEA-Sulfite, Methacrylic Acid/Sodium Acrylamidomethyl Propane Sulfonate Copolymer, Methacryloyl Ethyl Betaine/Acrylates Copolymer, Octylacrylamide/Acrylates/Butylaminoethyl Methacrylate Copolymer, PEG/PPG-25/25 Dimethicone/Acrylates Copolymer, PEG-8/SMDI Copolymer, Polyacrylamide, Polyacrylate-6, Polybeta-Alanine/Glutaric Acid Crosspolymer, Polybutylene Terephthalate, Polyester-1, Polyethylacrylate, Polyethylene Terephthalate, Polymethacryloyl Ethyl Betaine, Polypentaerythrityl Terephthalate, Polyperfluoroperhydrophenanthrene, Polyquaternium-1, Polyquaternium-2, Polyquaternium-4, Polyquaternium-5, Polyquaternium-6, Polyquaternium-7, Polyquaternium-8, Polyquaternium-9, Polyquaternium-10, Polyquaternium-11, Polyquaternium-12, Polyquaternium-13, Polyquaternium-14, Polyquaternium-15, Polyquaternium-16, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-30, Polyquaternium-31, Polyquaternium-32, Polyquaternium-33, Polyquaternium-34, Polyquaternium-35, Polyquaternium-36, Polyquaternium-37, Polyquaternium-39, Polyquaternium-45, Polyquaternium-46, Polyquaternium-47, Polyquaternium-48, Polyquaternium-49, Polyquaternium-50, Polyquaternium-55, Polyquaternium-56, Polysilicone-9, Polyurethane-1, Polyurethane-6, Polyurethane-10, Polyvinyl Acetate, Polyvinyl Butyral, Polyvinylcaprolactam, Polyvinylformamide, Polyvinyl Imidazolinium Acetate, Polyvinyl Methyl Ether, Potassium Butyl Ester of PVM/MA Copolymer, Potassium Ethyl Ester of PVM/MA Copolymer, PPG-70 Polyglyceryl-10 Ether, PPG-12/SMDI Copolymer, PPG-51/SMDI Copolymer, PPG-10 Sorbitol, PVM/MA Copolymer, PVP, PVP/VA/Itaconic Acid Copolymer, PVP/VA/Vinyl Propionate Copolymer, Rhizobian Gum, Rosin Acrylate, Shellac, Sodium Butyl Ester of PVM/MA Copolymer, Sodium Ethyl Ester of PVM/MA Copolymer, Sodium Polyacrylate, Sterculia Urens Gum, Terephthalic Acid/Isophthalic Acid/Sodium Isophthalic Acid Sulfonate/Glycol Copolymer, Trimethylolpropane Triacrylate, Trimethylsiloxysilylcarbamoyl Pullulan, VA/Crotonates Copolymer, VA/Crotonates/Methacryloxybenzophenone-1 Copolymer, VA/Crotonates/Vinyl Neodecanoate Copolymer, VA/Crotonates/Vinyl Propionate Copolymer, VA/DBM Copolymer, VA/Vinyl Butyl Benzoate/Crotonates Copolymer, Vinylamine/Vinyl Alcohol Copolymer, Vinyl Caprolactam/VP/Dimethylaminoethyl Methacrylate Copolymer, VP/Acrylates/Lauryl Methacrylate Copolymer, VP/Dimethylaminoethylmethacrylate Copolymer, VP/DMAPA Acrylates Copolymer, VP/Hexadecene Copolymer, VP/VA Copolymer, VP/Vinyl Caprolactam/DMAPA Acrylates Copolymer, Yeast Palmitate and Styrene/VP Copolymer. Cellulose ethers such as hydroxypropyl cellulose, hydroxyethyl cellulose, and methyl hydroxypropyl cellulose are also suitable.

Homopolyacrylic acid (INCI: Carbomer), which is commercially available under the name Carbopol® in various forms, is suitable as a firming compound.

Preferably, the fixing compound a polymer containing vinylpyrrolidone. Particularly preferably, the firming compound comprises a polymer selected from the group consisting of polyvinylpyrrolidone (PVP), vinylpyrrolidone-vinyl acetate copolymer (VP/VA copolymer), vinyl caprolactam/VP/dimethylaminoethyl methacrylate copolymer (INCI), VP/DMAPA acrylates copolymer (INCI), and mixtures thereof.

Another preferred solidifying compound is octylacrylamide/acrylates/butylaminoethyl methacrylate copolymer (INCI), which is marketed under the name “Amphomer®” by Akzo Nobel.

Accordingly, it is particularly preferred that the firming compound comprises a synthetic polymer selected from the group consisting of polyvinylpyrrolidone (PVP), vinylpyrrolidone-vinyl acetate copolymer (VP/VA copolymer), vinyl caprolactam/VP/dimethylaminoethyl methacrylate copolymer (INCI), VP/DMAPA acrylates copolymer (INCI), octylacrylamide/acrylates/butylaminoethyl methacrylate copolymer (INCI), and mixtures thereof.

According to further preferred embodiments, the cosmetic composition comprises at least one cationic surfactant of formula (V),

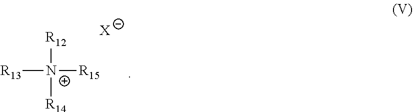

wherein
R₁₂, R₁₃, R₁₄ independently represent a C1-C6 alkyl group, a C2-C6 alkenyl group or a C2-C6 hydroxyalkyl group,
R₁₅ represents a C8-C28 alkyl group, preferably a C10-C22 alkyl group, and
X— stands for a physiologically compatible anion,
and/or the cosmetic composition comprises at least one cationic surfactant of the formula (VI),

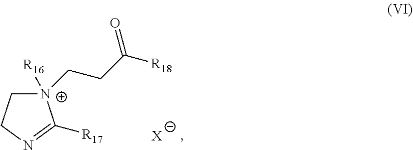

wherein
R₁₆ stands for a C1-C6 alkyl group
R₁₇, R₁₈ are independently a C7-C27 alkyl group, preferably a C10-C22 alkyl group, and
X— stands for a physiologically compatible anion,
and/or the cosmetic composition contains at least one cationic surfactant of the formula (VII),

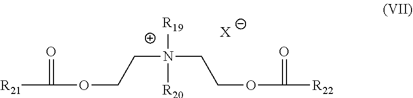

wherein
R₁₉, R₂₀ independently represent a C1-C6 alkyl group, a C2-C6 alkenyl group or a C2-C6 hydroxyalkyl group,
R₂₁, R₂₂ are independently a C7-C27 alkyl group, preferably a C10-C22 alkyl group, and
X— stands for a physiologically compatible anion,
and/or the cosmetic composition contains at least one cationic surfactant of the formula (VIII),

NR₂₃R₂₄R₂₅ (VIII)

wherein
R₂₃, R₂₄ independently represent a C1-C6 alkyl group, a C2-C6 alkenyl group or a C2-C6 hydroxyalkyl group, and
R₂₅ represents a C8-C28 alkyl group, preferably a C10-C22 alkyl group.

The cationic surfactants of formula (VIII) are amine derivatives, so-called pseudoquats. The organic radicals R₂₃, R₂₄ and R₂₅ are bonded directly to the nitrogen atom. In the acidic pH range, these are cationized, i.e. the nitrogen atom is then protonated. The physiologically compatible counterions are suitable as counterions. Steamidopropyl dimethylamine is particularly preferred among the cationic surfactants of formula (VIII).

According to a preferred embodiment, the amount of cationic surfactant is about 0.1 to about 30% by weight, preferably from about 0.5 to about 20% by weight, more preferably from about 1 to about 10% by weight, based on the total weight of the cosmetic composition.

According to a preferred embodiment, the cationic surfactant comprises a hydrophobic head group with a cationic charge and one or two hydrophobic end portions, wherein the hydrophobic end portion(s) represent straight-chain or branched, saturated or mono- or polyunsaturated alkyl groups, preferably having a chain length of C6 to C30, more preferably C8 to C26, particularly preferably C10 to C22. According to another preferred embodiment, the cationic surfactant has an ester function, an ether function, a ketone function, an alcohol function, or an amide function.

According to a preferred embodiment, the cosmetic agent further comprises a nonionic surfactant. This preferably comprises a nonionic surfactant selected from the group consisting of:

Alkyl glucamide comprising a saturated or unsaturated, branched or unbranched C₆ to C₂₂, preferably C₁₀ to C₁₈, more preferably C₁₂ to C₁₆ alkyl group,

An alkyl fructoside comprising a saturated or unsaturated, branched or unbranched C₆ to C₂₂, preferably C₁₀ to C₁₈, more preferably C₁₂ to C₁₆ alkyl group, alkyl glucoside comprising a saturated or unsaturated, branched or unbranched C₆ to C₂₂, preferably C₁₀ to C₁₈, more preferably C₁₂ to C₁₆ alkyl group, and

Alkyl alcohol alkoxylate of the formula R₁₀(OR₁₁)ₘOH, in which R₁₀ represents a linear or branched C₆ to C₂₂, preferably C₁₀ to C₁₈, more preferably C₁₂ to C₁₆ alkyl group, R₁₁ represents a C₂ to C₄, preferably a C₂ alkyl group, and m represents 1 to 10, preferably 2 to 6, more preferably 2 to 6.

According to preferred embodiments, one or more anionic surfactants are included in the cosmetic agent, which is preferably selected from the group consisting of:

straight-chain or branched, saturated or mono- or polyunsaturated alkylsulfonates having 8 to 24, preferably 12 to 22, more preferably 16 to 18, carbon atoms,

linear alpha-olefin sulfonates having 8 to 24, preferably 12 to 22, more preferably 16 to 18, carbon atoms,

Alkyl sulfates and alkyl polyglycol ether sulfates of the formula R₉—O—(CH₂—CH₂O)n-SO₃X, in which R₉ is preferably a straight-chain or branched, saturated or mono- or polyunsaturated alkyl or alkenyl radical having 8 to 24, preferably 12 to 22, more preferably 16 to 18 carbon atoms, n is 0 or 1 to 12, more preferably 2 to 4, and X is an alkali metal or alkaline earth metal ion or protonated triethanolamine or the ammonium ion,

straight-chain or branched, saturated or mono- or polyunsaturated alkylcarboxylic acids containing 8 to 24, preferably 12 to 22, more preferably 16 to 18 carbon atoms,

straight-chain or branched, saturated or mono- or polyunsaturated alkyl phosphates containing 8 to 24, preferably 12 to 22, more preferably 16 to 18 carbon atoms,

Alkyl isethionate whose alkyl group is selected from a branched or unbranched C₆ to C₂₂, preferably C₁₀ to C₁₈, more preferably C₁₂ to C₁₆ alkyl group, in particular sodium cocoyl isethionate,

Alkyl glycoside carboxylic acids whose alkyl group is selected from a branched or unbranched C₆ to C₂₂, preferably C₁₀ to C₁₈, more preferably C₁₂ to C₁₆ alkyl group,

Alkyl sulfosuccinates, the two alkyl groups of which are selected from identical or different, branched or unbranched C₂ to C₁₂, preferably C₄ to C₁₉, more preferably C₆ to C₈ alkyl groups,

Alkyl taurates, whose alkyl group is selected from a branched or unbranched C₆ to C₂₂, preferably C₁₀ to C₁₈, more preferably C₁₂ to C₁₆ alkyl group,

Alkyl sarcosinates, whose alkyl group is selected from a branched or unbranched C₆ to C₂₂, preferably C₁₀ to C₁₈, more preferably C₁₂ to C₁₆ alkyl group, and

Sulfonates of unsaturated fatty acids with 8 to 24, preferably 12 to 22, more preferably 16 to 18 C atoms and 1 to 6 double bonds,

wherein the counterion of the anionic surfactant is an alkali or alkaline earth metal ion or a protonated triethanolamine or the ammonium ion.

Particularly preferred anionic surfactants are straight-chain or branched alkyl ether sulfates containing an alkyl radical with 8 to 18 and in particular with 10 to 16 carbon atoms and 1 to 6 and in particular 2 to 4 ethylene oxide units. Very preferably, the surfactant mixture of anionic and amphoteric/zwitterionic surfactants contains sodium lauryl ether sulfate (INCI: sodium laureth sulfate) and very preferably sodium lauryl ether sulfate with 2 ethylene oxide units.

Amphoteric surfactants, also known as zwitterionic surfactants, are surface-active compounds that contain at least one quaternary ammonium group and at least one —COO— or —SO₃— group in the molecule. Amphoteric/zwitterionic surfactants also include surface-active compounds which, in addition to a C8-C24 alkyl or acyl group, contain at least one free amino group and at least one —COOH or —SO₃H group and are capable of forming internal salts.

According to a preferred embodiment, the amphoteric surfactants in the cosmetic agent are selected from the group consisting of:

alkyl betaine comprising at least one saturated or unsaturated, branched or unbranched C₆ to C₂₂, preferably C₁₀ to C₁₈, more preferably C₁₂ to C₁₆ alkyl group,

alkyl amphodiacetate or alkyl amphodiacetate comprising a saturated or unsaturated, branched or unbranched C₆ to C₂₂, preferably C₁₀ to C₁₈, more preferably C₁₂ to C₁₆ alkyl group, with an alkali or alkaline earth metal counterion, and

alkylamidopropyl betaine comprising at least one saturated or unsaturated, branched or unbranched C₆ to C₂₂, preferably C₁₀ to C₁₈, more preferably C₁₂ to C₁₆ alkyl group.

Particularly suitable amphoteric/zwitterionic surfactants include those known under the INCI designation cocamidopropyl betaine and disodium cocoamphodiacetate.

According to a preferred embodiment, the nonionic surfactant is selected from the group consisting of:

Alkyl glucamide comprising a saturated or unsaturated, branched or unbranched C₆ to C₂₂, preferably C₁₀ to C₁₈, more preferably C₁₂ to C₁₆ alkyl group,

An alkyl fructoside comprising a saturated or unsaturated, branched or unbranched C₆ to C₂₂, preferably C₁₀ to C₁₈, more preferably C₁₂ to C₁₆ alkyl group,

Alkylglucosid comprising a saturated or unsaturated, branched or unbranched C₆ to C₂₂, preferably C₁₀ to C₁₈, more preferably C₁₂ to C₁₆ alkyl group,

Alkyl alcohol alkoxylate of the formula R₁₀(OR₁₁)ₘOH, in which R₁₀ represents a linear or branched C₆ to C₂₂, preferably C₁₀ to C₁₈, more preferably C₁₂ to C₁₆ alkyl group, R₁₁ represents a C₂ to C₄, preferably a C₂ alkyl group, and ₘ represents 1 to 10, preferably 2 to 6, more preferably 2 to 6, and

Alkyl esters of the formula R₁₂COOR₁₃, in which R₁₂ represents a linear or branched C₆ to C₂₂, preferably C₁₀ to C₁₈, more preferably C₁₂ to C₁₆ alkyl group, R₁₃ represents a C₁ to C₄, preferably a C₂ alkyl group.

According to a preferred embodiment, the cosmetic agent contains two structurally different surfactants. It is particularly preferred that the cosmetic agent contains two structurally different surfactants from one another, preferably the cosmetic agent contains two structurally different cationic surfactants from one another, or the cosmetic agent contains a cationic surfactant and a nonionic surfactant.

The cosmetic compound may contain, in addition or as an alternative to a synthetic polymer, at least one natural or synthetic wax having a melting point above 37° C. as a firming compound.

Natural or synthetic waxes may be solid kerosenes or isoparaffins, vegetable waxes such as candelilla wax, carnauba wax, esparto grass wax, Japan wax, cork wax, sugar cane wax, ouricury wax, montan wax, sunflower wax, fruit waxes and animal waxes such as beeswaxes and other insect waxes, Whale wax, shellac wax, wool wax and brushing grease, furthermore mineral waxes, such as ceresin and ozokerite or petrochemical waxes, such as petrolatum, kerosene waxes, microwaxes of polyethylene or polypropylene and polyethylene glycol waxes can be used. It may be advantageous to use hydrogenated or cured waxes. Chemically modified waxes, in particular hard waxes such as montan ester waxes, sasol waxes and hydrogenated jojoba waxes, can also be used.

Also suitable for use in the cosmetic agents, in addition to the mandatory components, are the triglycerides of saturated and optionally hydroxylated C16-30 fatty acids, such as hydrogenated triglyceride fats (hydrogenated palm oil, hydrogenated coconut oil, hydrogenated castor oil), glyceryl tribehenate or glyceryl tri-12-hydroxystearate.

The wax components can also be selected from the group of esters of saturated, unbranched alkanecarboxylic acids having a chain length of 22 to 44 carbon atoms and saturated, unbranched alcohols having a chain length of 22 to 44 carbon atoms, provided that the wax component or the totality of wax components are solid at room temperature. Silicone waxes, for example stearyltrimethylsilane/stearyl alcohol, may also be beneficial.

Natural, chemically modified and synthetic waxes can be used alone or in combination. Thus, several waxes can also be used. Furthermore, a number of wax compounds, possibly mixed with other additives, are also commercially available. The products sold under the designations “Special Wax 7686 OE” (a mixture of cetyl palmitate, beeswax, microcrystalline wax and polyethylene with a melting range of about 73-75° C.; manufacturer: Kahl & Co), Polywax® GP 200 (a mixture of stearyl alcohol and polyethylene glycol stearate with a melting point of about 47-51° C.; manufacturer: Croda) and “Softceresin® FL 400” (a vaseline/vaseline oil/wax mixture with a melting point of about 50-54° C.; manufacturer: Parafluid Mineral Oil Company) are examples of mixtures that can be used.

Preferably, the wax is selected from carnauba wax (INCI: Copernicia Cerifera Cera) Beeswax (INCI: Beeswax), petrolatum (INCI), microcrystalline wax and especially mixtures thereof.

Preferred blends include the combination of carnauba wax (INCI: Copernicia Cerifera Cera), petrolatum and microcrystalline wax or the combination of beeswax (INCI: Beeswax) and Petrolatum.

Wax or the wax components should be solid at 25° C. and should melt in the range >37° C.

The composition for treating a keratinous material contains a firming compound preferably in a total amount of about 0.5 to about 50% by weight, preferably from about 1 to about 40% by weight, more preferably from about 1.5 to about 30% by weight, even more preferably from about 2 to about 25% by weight, based on the total weight of the cosmetic composition.

Other suitable ingredients include nonionic polymers, anionic polymers, (further) cationic polymers, waxes, oligopetides, vitamins, provitamins, vitamin precursors, betaines, bioquinones, purine (derivatives), care substances, plant extracts, silicones, ester oils, UV light protection filters, structuring agents, thickeners, electrolytes, pH-adjusting agents, swelling agents, colorants, anti-dandruff agents, complexing agents, opacifiers, pearlescent agents, pigments, stabilizing agents, propellants, antioxidants, perfume oils and/or preservatives.

In preferred embodiments 1 to 104, the preferred organic silicon compounds are combined with preferred components b) in a cosmetic agent as contemplated herein.

| | Silane compound | other ingredients |
|---|---|---|
| 1 | (3-Aminopropyl)trimethoxysilane | Cystine Bis-PG-Propyl |
| | | Silanetriol (INCI) |
| 2 | (3-Aminopropyl)triethoxysilan | Cystine Bis-PG-Propyl |
| | | Silanetriol (INCI) |
| 3 | (2-Aminoethyl)trimethoxysilane | Cystine Bis-PG-Propyl |
| | | Silanetriol (INCI) |
| 4 | (2-Aminoethyl)triethoxysilane | Cystine Bis-PG-Propyl |
| | | Silanetriol (INCI) |
| 5 | (3-Dimethylaminopropyl) | Cystine Bis-PG-Propyl |
| | trimethoxysilane | Silanetriol (INCI) |
| 6 | (3-Dimethylaminopropyl) | Cystine Bis-PG-Propyl |
| | triethoxysilane | Silanetriol (INCI) |
| 7 | (2-Dimethylaminoethyl) | Cystine Bis-PG-Propyl |
| | trimethoxysilane | Silanetriol (INCI) |
| 8 | (2-Dimethylaminoethyl) | Cystine Bis-PG-Propyl |
| | triethoxysilane | Silanetriol (INCI) |
| 9 | 3-(trimethoxysilyl)-N-[3- | Cystine Bis-PG-Propyl |
| | (trimethoxysilyl)propyl]- | Silanetriol (INCI) |
| | 1-propanamine | |
| 10 | 3-(triethoxysilyl)-N-[3- | Cystine Bis-PG-Propyl |
| | (triethoxysilyl)propyl]- | Silanetriol (INCI) |
| | 1-propanamine | |
| 11 | N-methyl-3-(trimethoxysilyl)-N-[3- | Cystine Bis-PG-Propyl |
| | (trimethoxysilyl)propyl]- | Silanetriol (INCI) |
| | 1-propanamine | |
| 12 | N-Methyl-3-(triethoxysilyl)-N-[3- | Cystine Bis-PG-Propyl |
| | (triethoxysilyl)propyl]- | Silanetriol (INCI) |
| | 1-propanamine | |
| 13 | 2-[Bis[3-(trimethoxysilyl) | Cystine Bis-PG-Propyl |
| | propyl]aminol-ethanol | Silanetriol (INCI) |
| 14 | 2-[bis[3-(triethoxysilyl) | Cystine Bis-PG-Propyl |
| | propyl]amino]ethanol | Silanetriol (INCI) |
| 15 | 3-(trimethoxysilyl)-N,N-bis[3- | Cystine Bis-PG-Propyl |
| | (trimethoxysilyl)propyl]- | Silanetriol (INCI) |
| | 1-propanamine | |
| 16 | 3-(triethoxysilyl)-N,N-bis[3- | Cystine Bis-PG-Propyl |
| | (triethoxysilyl)propyl]- | Silanetriol (INCI) |
| | 1-propanamine | |
| 17 | N1,N1-Bis[3-(trimethoxysilyl) | Cystine Bis-PG-Propyl |
| | propyl]-1,2-ethanediamine | Silanetriol (INCI) |
| 18 | N1,N1-Bis[3-(triethoxysilyl) | Cystine Bis-PG-Propyl |
| | propyl]-1,2-ethanediamine | Silanetriol (INCI) |
| 19 | N,N-Bis[3-(trimethoxysilyl) | Cystine Bis-PG-Propyl |
| | propyl]-2-propen-1-amine | Silanetriol (INCI) |
| 20 | N,N-Bis[3-(triethoxysilyl) | Cystine Bis-PG-Propyl |
| | propyl]-2-propen-1-amine | Silanetriol (INCI) |
| 21 | Methyltrimethoxysilane | Cystine Bis-PG-Propyl |
| | | Silanetriol (INCI) |
| 22 | Methyltriethoxysilane | Cystine Bis-PG-Propyl |
| | | Silanetriol (INCI) |
| 23 | Ethyltrimethoxysilane | Cystine Bis-PG-Propyl |
| | | Silanetriol (INCI) |
| 24 | Ethyltriethoxysilane | Cystine Bis-PG-Propyl |
| | | Silanetriol (INCI) |
| 25 | Octyltrimethoxysilane | Cystine Bis-PG-Propyl |
| | | Silanetriol (INCI) |
| 26 | Octyltriethoxysilane | Cystine Bis-PG-Propyl |
| | | Silanetriol (INCI) |
| 27 | Dodecyltrimethoxysilane | Cystine Bis-PG-Propyl |
| | | Silanetriol (INCI) |
| 28 | Dodecyltriethoxysilane | Cystine Bis-PG-Propyl |
| | | Silanetriol (INCI) |
| 29 | (3-Aminopropyl)trimethoxysilane | Hydrolyzed Wheat Protein |
| | | PG-Propyl Silanetriol (INCI) |
| 30 | (3-Aminopropyl)triethoxysilan | Hydrolyzed Wheat Protein |
| | | PG-Propyl Silanetriol (INCI) |
| 31 | (2-Aminoethyl)trimethoxysilane | Hydrolyzed Wheat Protein |
| | | PG-Propyl Silanetriol (INCI) |
| 32 | (2-Aminoethyl)triethoxysilane | Hydrolyzed Wheat Protein |
| | | PG-Propyl Silanetriol (INCI) |
| 33 | (3-Dimethylaminopropyl) | Hydrolyzed Wheat Protein |
| | trimethoxysilane | PG-Propyl Silanetriol (INCI) |
| 34 | (3-Dimethylaminopropyl) | Hydrolyzed Wheat Protein |
| | triethoxysilane | PG-Propyl Silanetriol (INCI) |
| 35 | (2-Dimethylaminoethyl) | Hydrolyzed Wheat Protein |
| | trimethoxysilane | PG-Propyl Silanetriol (INCI) |
| 36 | (2-Dimethylaminoethyl) | Hydrolyzed Wheat Protein |
| | triethoxysilane | PG-Propyl Silanetriol (INCI) |
| 37 | 3-(trimethoxysilyl)-N-[3- | Hydrolyzed Wheat Protein |
| | (trimethoxysilyl)propyl]- | PG-Propyl Silanetriol (INCI) |
| | 1-propanamine | |
| 38 | 3-(triethoxysilyl)-N-[3- | Hydrolyzed Wheat Protein |
| | (triethoxysilyl)propyl]- | PG-Propyl Silanetriol (INCI) |
| | 1-propanamine | |
| 39 | N-methyl-3-(trimethoxysilyl)- | Hydrolyzed Wheat Protein |
| | N-[3-(trimethoxysilyl)propyl]- | PG-Propyl Silanetriol (INCI) |
| | 1-propanamine | |
| 40 | N-Methyl-3-(triethoxysilyl)- | Hydrolyzed Wheat Protein |
| | N[3-(triethoxysilyl)propyl]- | PG-Propyl Silanetriol (INCI) |
| | 1-propanamine | |
| 41 | 2-[Bis[3-(trimethoxysilyl) | Hydrolyzed Wheat Protein |
| | propyl]amino]-ethanol | PG-Propyl Silanetriol (INCI) |
| 42 | 2-[bis[3-(triethoxysilyl) | Hydrolyzed Wheat Protein |
| | propyl]amino]ethanol | PG-Propyl Silanetriol (INCI) |
| 43 | 3-(trimethoxysilyl)-N,N-bis | Hydrolyzed Wheat Protein |
| | [3-(trimethoxysilyl)propyl]- | PG-Propyl Silanetriol (INCI) |
| | 1-propanamine | |
| 44 | 3-(triethoxysilyl)-N,N-bis | Hydrolyzed Wheat Protein |
| | [3-(triethoxysilyl)propyl]- | PG-Propyl Silanetriol (INCI) |
| | 1-propanamine | |
| 45 | N1,N1-Bis[3-(trimethoxysilyl) | Hydrolyzed Wheat Protein |
| | propyl]-1,2-ethanediamine | PG-Propyl Silanetriol (INCI) |
| 46 | N1,N1-Bis[3-(triethoxysilyl) | Hydrolyzed Wheat Protein |
| | propyl]-1,2-ethanediamine | PG-Propyl Silanetriol (INCI) |
| 47 | N,N-Bis[3-(trimethoxysilyl) | Hydrolyzed Wheat Protein |
| | propyl]-2-propen-1-amine | PG-Propyl Silanetriol (INCI) |
| 48 | N,N-Bis[3-(triethoxysilyl) | Hydrolyzed Wheat Protein |
| | propyl]-2-propen-1-amine | PG-Propyl Silanetriol (INCI) |
| 49 | Methyltrimethoxysilane | Hydrolyzed Wheat Protein |
| | | PG-Propyl Silanetriol (INCI) |
| 50 | Methyltriethoxysilane | Hydrolyzed Wheat Protein |
| | | PG-Propyl Silanetriol (INCI) |
| Si | Ethyltrimethoxysilane | Hydrolyzed Wheat Protein |
| | | PG-Propyl Silanetriol (INCI) |
| 52 | Ethyltriethoxysilane | Hydrolyzed Wheat Protein |
| | | PG-Propyl Silanetriol (INCI) |
| 53 | Octyltrimethoxysilane | Hydrolyzed Wheat Protein |
| | | PG-Propyl Silanetriol (INCI) |
| 54 | Octyltriethoxysilane | Hydrolyzed Wheat Protein |
| | | PG-Propyl Silanetriol (INCI) |
| 55 | Dodecyltrimethoxysilane | Hydrolyzed Wheat Protein |
| | | PG-Propyl Silanetriol (INCI) |
| 56 | Dodecyltriethoxysilane | Hydrolyzed Wheat Protein |
| | | PG-Propyl Silanetriol (INCI) |
| 57 | Propyltrimethoxysilane | Cystine Bis-PG-Propyl |
| | | Silanetriol (INCI) |
| 58 | Propyltriethoxysilane | Cystine Bis-PG-Propyl |
| | | Silanetriol (INCI) |
| 59 | Hexyltrimethoxysilane | Cystine Bis-PG-Propyl |
| | | Silanetriol (INCI) |
| 60 | Hexyltriethoxysilane | Cystine Bis-PG-Propyl |
| | | Silanetriol (INCI) |
| 61 | Octyltrimethoxysilane | Cystine Bis-PG-Propyl |
| | | Silanetriol (INCI) |
| 62 | Octyltriethoxysilane | Cystine Bis-PG-Propyl |
| | | Silanetriol (INCI) |
| 63 | Dodecyltrimethoxysilane | Cystine Bis-PG-Propyl |
| | | Silanetriol (INCI) |
| 64 | Dodecyltriethoxysilane | Cystine Bis-PG-Propyl |
| | | Silanetriol (INCI) |
| 65 | Octadecyltriethoxysilane | Cystine Bis-PG-Propyl |
| | | Silanetriol (INCI) |
| 66 | Octadecyltriethoxysilane | Cystine Bis-PG-Propyl |
| | | Silanetriol (INCI) |
| 67 | (3-Aminopropyl)triethoxysilan + | Cystine Bis-PG-Propyl |
| | Methyltrimethoxysilan | Silanetriol (INCI) |
| 68 | (3-Aminopropyl)triethoxysilane + | Cystine Bis-PG-Propyl |
| | Methyltriethoxysilane | Silanetriol (INCI) |
| 69 | (3-Aminopropyl)triethoxysilane + | Cystine Bis-PG-Propyl |
| | Ethyltrimethoxysilane | Silanetriol (INCI) |
| 70 | (3-Aminopropyl)triethoxysilane + | Cystine Bis-PG-Propyl |
| | Ethyltriethoxysilane | Silanetriol (INCI) |
| 71 | (3-Aminopropyl)triethoxysilane + | Cystine Bis-PG-Propyl |
| | Propyltrimethoxysilane | Silanetriol (INCI) |
| 72 | (3-Aminopropyl)triethoxysilane + | Cystine Bis-PG-Propyl |
| | Propyltriethoxysilane | Silanetriol (INCI) |
| 73 | (3-Aminopropyl)triethoxysilane + | Cystine Bis-PG-Propyl |
| | Hexyltrimethoxysilane | Silanetriol (INCI) |
| 74 | (3-Aminopropyl)triethoxysilane + | Cystine Bis-PG-Propyl |
| | Hexyltriethoxysilane | Silanetriol (INCI) |
| 75 | (3-Aminopropyl)triethoxysilane + | Cystine Bis-PG-Propyl |
| | Octyltrimethoxysilane | Silanetriol (INCI) |
| 76 | (3-Aminopropyl)triethoxysilane + | Cystine Bis-PG-Propyl |
| | Octyltriethoxysilane | Silanetriol (INCI) |
| 77 | (3-Aminopropyl)triethoxysilane + | Cystine Bis-PG-Propyl |
| | Dodecyltrimethoxysilane | Silanetriol (INCI) |
| 78 | (3-Aminopropyl)triethoxysilane + | Cystine Bis-PG-Propyl |
| | Dodecyltriethoxysilane | Silanetriol (INCI) |
| 79 | (3-Aminopropyl)triethoxysilane + | Cystine Bis-PG-Propyl |
| | octyldecyltrimethoxysilane | Silanetriol (INCI) |
| 80 | (3-Aminopropyl)triethoxysilane + | Cystine Bis-PG-Propyl |
| | Octyldecyltriethoxysilane | Silanetriol (INCI) |
| 81 | Propyltrimethoxysilane | Hydrolyzed Wheat Protein |
| | | PG-Propyl Silanetriol (INCI) |
| 82 | Propyltriethoxysilane | Hydrolyzed Wheat Protein |
| | | PG-Propyl Silanetriol (INCI) |
| 83 | Hexyltrimethoxysilane | Hydrolyzed Wheat Protein |
| | | PG-Propyl Silanetriol (INCI) |
| 84 | Hexyltriethoxysilane | Hydrolyzed Wheat Protein |
| | | PG-Propyl Silanetriol (INCI) |
| 85 | Octyltrimethoxysilane | Hydrolyzed Wheat Protein |
| | | PG-Propyl Silanetriol (INCI) |
| 86 | Octyltriethoxysilane | Hydrolyzed Wheat Protein |
| | | PG-Propyl Silanetriol (INCI) |
| 87 | Dodecyltrimethoxysilane | Hydrolyzed Wheat Protein |
| | | PG-Propyl Silanetriol (INCI) |
| 88 | Dodecyltriethoxysilane | Hydrolyzed Wheat Protein |
| | | PG-Propyl Silanetriol (INCI) |
| 89 | Octadecyltriethoxysilane | Hydrolyzed Wheat Protein |
| | | PG-Propyl Silanetriol (INCI) |
| 90 | Octadecyltriethoxysilane | Hydrolyzed Wheat Protein |
| | | PG-Propyl Silanetriol (INCI) |
| 91 | (3-Aminopropyl)triethoxysilan + | Hydrolyzed Wheat Protein |
| | Methyltrimethoxysilan | PG-Propyl Silanetriol (INCI) |
| 92 | (3-Aminopropyl)triethoxysilane + | Hydrolyzed Wheat Protein |
| | Methyltriethoxysilane | PG-Propyl Silanetriol (INCI) |
| 93 | (3-Aminopropyl)triethoxysilane + | Hydrolyzed Wheat Protein |
| | Ethyltrimethoxysilane | PG-Propyl Silanetriol (INCI) |
| 94 | (3-Aminopropyl)triethoxysilane + | Hydrolyzed Wheat Protein |
| | Ethyltriethoxysilane | PG-Propyl Silanetriol (INCI) |
| 95 | (3-Aminopropyl)triethoxysilane + | Hydrolyzed Wheat Protein |
| | Propyltrimethoxysilane | PG-Propyl Silanetriol (INCI) |
| 96 | (3-Aminopropyl)triethoxysilane + | Hydrolyzed Wheat Protein |
| | Propyltriethoxysilane | PG-Propyl Silanetriol (INCI) |
| 97 | (3-Aminopropyl)triethoxysilane + | Hydrolyzed Wheat Protein |
| | Hexyltrimethoxysilane | PG-Propyl Silanetriol (INCI) |
| 98 | (3-Aminopropyl)triethoxysilane + | Hydrolyzed Wheat Protein |
| | Hexyltriethoxysilane | PG-Propyl Silanetriol (INCI) |
| 99 | (3-Aminopropyl)triethoxysilane + | Hydrolyzed Wheat Protein |
| | Octyltrimethoxysilane | PG-Propyl Silanetriol (INCI) |
| 100 | (3-Aminopropyl)triethoxysilane + | Hydrolyzed Wheat Protein |
| | Octyltriethoxysilane | PG-Propyl Silanetriol (INCI) |
| 101 | (3-Aminopropyl)triethoxysilane + | Hydrolyzed Wheat Protein |
| | Dodecyltrimethoxysilane | PG-Propyl Silanetriol (INCI) |
| 102 | (3-Aminopropyl)triethoxysilane + | Hydrolyzed Wheat Protein |
| | Dodecyltriethoxysilane | PG-Propyl Silanetriol (INCI) |
| 103 | (3-Aminopropyl)triethoxysilane + | Hydrolyzed Wheat Protein |
| | octyldecyltrimethoxysilane | PG-Propyl Silanetriol (INCI) |
| 104 | (3-Aminopropyl)triethoxysilane + | Hydrolyzed Wheat Protein |
| | Octyldecyltriethoxysilane | PG-Propyl Silanetriol (INCI) |

The combinations in the above table represent combinations of active ingredients that are combined in cosmetic agents with other components described above.

The active ingredient combination of at least one organic silicon compound and another component selected from the group consisting of an amino acid, an amino acid-derived polymer, a silanized amino acid, and an amino acid-derived silanized polymer may already be present in the composition for treating a keratinous material. In this embodiment, the agent for treating a keratinous material is already distributed in a ready-to-use form. In order to provide a formulation that is as stable as possible during storage, the agent itself is preferably packaged with low or no water.

Alternatively, at least one organic silicon compound is added a maximum of 12 hours, preferably a maximum of 6 hours, more preferably a maximum of 3 hours, even more preferably a maximum of 1 hour prior to application of the keratinous material treatment agent to a base comprising all of the ingredients of the keratinous material treatment agent except the at least one organic silicon compound.

Furthermore, alternatively, the organic silicon compound and another component b) are added to a cosmetic product only shortly before use, i.e. 1 minute to 12 hours, preferably from about 2 minutes to about 6 hours, particularly preferably from about 1 minute to about 3 hours, especially preferably from about 1 minute to about 1 hour.

In a further alternative, the AMEO or the bis(triethoxysilylpropyl)amine is added to an aqueous solution which is applied to the hair and, in a second step, an aqueous solution or a cosmetic agent containing the further component b) is applied to the hair.

For example, the user may first mix or shake an agent (α) comprising the organic silicon compound(s) with an agent (β) comprising the remaining ingredients of the agent for treating a keratinous material. The user can now apply this mixture of (α) and (β)—either directly after its preparation or after a short reaction time of 1 minute to 20 minutes—to the keratinous materials. The agent (β) may contain water, in particular water in an amount >30% by weight, based on the total weight of the agent for treating keratinous materials.

Another subject matter of the present application is the use of a cosmetic agent as contemplated herein for treating a keratinous material

for the care of keratinous material and/or for the hydrophobization of keratinous material.

With regard to further preferred embodiments of use, the same applies mutatis mutandis as to the cosmetic agents.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic agent for the treatment of a keratinous material, comprising
a) at least one organic silicon compound and
b) at least one other component selected from the group consisting of an amino acid, an amino acid-derived polymer, a silanized amino acid, and an amino acid-derived silanized polymer.

2. A cosmetic agent for treating a keratinous material according to claim 1, wherein the at least one organic silicon compound comprises a compound of the formula (I) and/or (II),
wherein in the organic silicon compound of formula (I)
R₁R₂N-L-Si(OR₃)ₐ(R₄)_{b} (I),
R₁, R₂ both represent a hydrogen atom,
L represents a linear, divalent C₁-C₆-alkylene group,
R₃, R₄ independently represent a methyl group or an ethyl group,
a stands for the number 3, and
b stands for the number 0, and
wherein in the organic silicon compound of formula (II)
(R₅O)_{c}(R₆)_{d}Si-(A)ₑ-[NR₇-(A′)]_{f}—[O-(A″)]_{g}—[NR₈-(A′″)]ₕ—Si(R₆′)_{d′}(OR₅′)_{c′} (II),
R₅, R_{5′}, R_{5″}, R₆, R_{6′} and R_{6″} stand independently for a C₁-C₆ alkyl group,
A, A′, A″, A′″ and A″″ independently represent a linear or branched divalent C₁-C₂₀ alkylene group, and
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III):
-(A″″)-Si(R₆″)_{d}″(OR₅″)_{c}″ (III),
where
c stands for an integer from 1 to 3,
d stands for the integer 3-c,
c′ stands for an integer from 1 to 3,
d′ stands for the integer 3-c′,
c″ stands for an integer from 1 to 3,
d″ stands for the integer 3-c″,
e stands for 0 or 1,
f stands for 0 or 1,
g stands for 0 or 1, and
h stands for 0 or 1,
with the proviso that at least one of the components of e, f, g and h is different from 0.

3. A cosmetic composition for treating a keratinous material according to claim 1,
wherein the agent for treating a keratinous material comprises at least one organic silicon compound of formula (I) selected from the group consisting of:
(3-Aminopropyl)trimethoxysilan
(3-Aminopropyl)triethoxysilan
(2-Aminoethyl)trimethoxysilan
(2-Aminoethyl)triethoxysilan
(3-Dimethylaminopropyl)trimethoxysilan
(3-Dimethylaminopropyl)triethoxysilan
(2-dimethylaminoethyl)trimethoxysilane, and
(2-Dimethylaminoethyl)triethoxysilan,
or wherein the agent for treating a keratinous material comprises at least one organic silicon compound of formula (II) selected from the group consisting of:
3-(trimethoxysilyl)-N-[3-(trimethoxysilyl)propyl]-1-propanamine,
3-(Triethoxysilyl)-N-[3-(triethoxysilyl)propyl]-1-propanamine,
N-methyl-3-(trimethoxysilyl)-N-[3-(trimethoxysilyl)propyl]-1-propanamine,
N-Methyl-3-(triethoxysilyl)-N-[3-(triethoxysilyl)propyl]-1-propanamine,
2-[Bis[3-(trimethoxysilyl)propyl]amino]-ethanol,
2-[bis[3-(triethoxysilyl)propyl]amino]ethanol,
3-(Trimethoxysilyl)-N,N-bis[3-(trimethoxysilyl)propyl]-1-propanamine,
3-(Triethoxysilyl)-N,N-bis[3-(triethoxysilyl)propyl]-1-propanamine,
N1,N1-bis[3-(trimethoxysilyl)propyl]-1,2-ethanediamine,
N1,N1-bis[3-(triethoxysilyl)propyl]-1,2-ethanediamine,
N,N-bis[3-(trimethoxysilyl)propyl]-2-propen-1-amine, and
N,N-bis[3-(triethoxysilyl)propyl]-2-propen-1-amine.

4. The cosmetic agent for treating a keratin material according to claim 1, wherein the organic silicon compound of formula (I) comprises from about 0.01 to about 10% by weight of the cosmetic agent, based on the total weight of the cosmetic agent, and/or wherein the organic silicon compound of formula (II) comprises from about 0.01 to about 10% by weight of the cosmetic agent, based on the total weight of the cosmetic agent,
and or wherein the organic silicon compound of formula (I) is 3-(triethoxysilyl)-N-[3-(triethoxysilyl)propyl]-1-propanamine and/or the organic silicon compound of formula (II) is (3-aminopropyl)triethoxysilane.

5. Cosmetic agent for treating a keratinous material according to claim 1, wherein the agent for treating a keratinous material comprises at least one organic silicon compound of formula (IV),
R₉Si(OR₁₀)ₖ(R₁₁)ₘ (IV),
which is selected from the group consisting of:
Methyltrimethoxysilane,
Methyltriethoxysilane,
Ethyltrimethoxysilane,
Ethyltriethoxysilane,
Propyltrimethoxysilane,
Propyltriethoxysilane,
Hexyltrimethoxysilane,
Hexyltriethoxysilane,
Octyltrimethoxysilane,
Octyltriethoxysilane,
Dodecyltrimethoxysilane,
Dodecyltriethoxysilane,
Octadecyltrimethoxysilane, and
Octadecyltriethoxysilane.

6. A cosmetic agent for treating a keratinous material according to claim 1, wherein the amino acid is selected from the group consisting of arginine, histidine, proline, cysteine and lanthionine, and/or wherein the silanized amino acid is a reaction product of an amino acid selected from the group consisting of arginine, histidine, proline, cystine and lanthionine with a silane,
and/or wherein the pH of the cosmetic agent is in the range from about 1.5 to about 8.

7. Cosmetic agent for the treatment of a keratinous material according to claim 1, wherein the polymer derived from the amino acid is a protein hydrolysate, preferably comprising elastin, collagen, keratin, silk, milk protein, soybean, almond, pea, rice, tomato, potato, wheat protein hydrolysates, or salts thereof, in particular keratin protein hydrolysates, wheat protein hydrolysates or salts thereof; and/or wherein
the silanized polymer derived from an amino acid is a silanized protein hydrolysate of formula (PH-I) 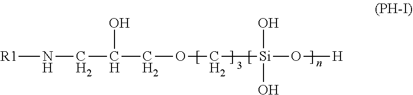
or the formula (PH-II) 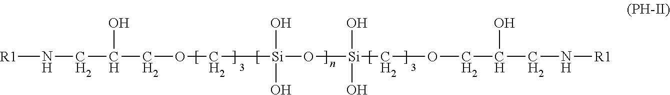
in which n is an integer from about 1 to about 100, and R1 represents a polypeptide chain of the protein hydrolysate used; and/or wherein
the further component b) is selected from the group consisting of Cystine Bis-PG-Propyl Silanetriol (INCI) and Hydrolyzed Wheat Protein PG-Propyl Silanetriol (INCI).

8. Cosmetic agent for the treatment of a keratinous material according to claim 1, wherein the one or more components b) selected from the group consisting of an amino acid, a polymer derived from an amino acid, a silanized amino acid and a silanized polymer derived from an amino acid are present in a total amount in the cosmetic composition which is from about 0.01 to about 10% by weight.

9. A cosmetic agent for treating a keratinous material according to claim 1, wherein the agent for treating a keratinous material comprises based on the total weight of the agent for treating a keratinous material:
from about 0.5 to about 3 weight % of at least one first organic silicon compound selected from the group consisting of (3-aminopropyl)trimethoxysilane, (3-aminopropyl)triethoxysilane, (2-aminoethyl)trimethoxysilane, (2-aminoethyl)triethoxysilane, (3-dimethylaminopropyl)trimethoxysilane, (3-dimethylaminopropyl)triethoxysilane (2-dimethylaminoethyl)trimethoxysilane and (2-dimethylaminoethyl)triethoxysilane, and
from about 3.2 to about 7% by weight of at least one second organic silicon compound selected from the group consisting of methyltrimethoxysilane, methyltriethoxysilane, ethyltrimethoxysilane, ethyltriethoxysilane, propyltrimethoxysilane, propyltriethoxysilane, octyltrimethoxysilane, octyltriethoxysilane, dodecyltrimethoxysilane and dodecyltriethoxysilane.

10. Use of a cosmetic agent for treating a keratinous material according to claim 1.
for temporary reshaping of keratinous material,
for the care of keratinous material and/or
for hydrophobizing the surface of keratinous material.

11. Cosmetic agent for treating a keratinous material according to claim 1, wherein the amino acid is selected from the group consisting of arginine, histidine, proline, cysteine and lanthionine.

12. Cosmetic agent for treating a keratinous material according to claim 1, wherein the silanized amino acid is a reaction product of an amino acid selected from the group consisting of arginine, histidine, proline, cystine and lanthionine with a silane.

13. Cosmetic agent for treating a keratinous material according to claim 1, wherein the pH of the cosmetic agent is from about 1.5 to about 8.

14. Cosmetic agent for treating a keratinous material according to claim 1, wherein the pH of the cosmetic agent is from about 2 to about 7.

15. Cosmetic agent for treating a keratinous material according to claim 1, wherein the pH of the cosmetic agent is from about 2.5 to about 6.

16. Cosmetic agent for treating a keratinous material according to claim 1, wherein the pH of the cosmetic agent is from about 3 to about 5.

17. Cosmetic agent for treating a keratinous material according to claim 1, wherein the one or more components b) selected from the group consisting of an amino acid, a polymer derived from an amino acid, a silanized amino acid and a silanized polymer derived from an amino acid are present in a total amount in the cosmetic composition of from about 0.25 to about 8% by weight.

18. Cosmetic agent for treating a keratinous material according to claim 1, wherein the one or more components b) selected from the group consisting of an amino acid, a polymer derived from an amino acid, a silanized amino acid and a silanized polymer derived from an amino acid are present in a total amount in the cosmetic composition of from about 0.5 to about 6% by weight.
